SEQUENCE LISTING (12 sequences)

SEQ ID NO: 1 - DNA, 27 bp, Synthetic construct
  source 1..27: mol_type = other DNA
tttttggcccgccgcagtatacaacct

SEQ ID NO: 2 - DNA, 47 bp, Synthetic construct
  source 1..47: mol_type = other DNA
gaccaataacaacgcggagagcgggtacatgcctggtccacgttttt

SEQ ID NO: 3 - DNA, 100 bp, Synthetic construct
  source 1..100: mol_type = other DNA
cgctctccgcgttgttattggtcaccggttgtggagccgcggttcgtaccttcggtgtagctcgggtatcgagcacggcatcgtggaccaggcatgtacc

SEQ ID NO: 4 - DNA, 66 bp, Synthetic construct
  source 1..66: mol_type = other DNA
cggcgggccgagcctcaagacccacaattttttcgaccgaaaaatcgattgtgtggttgtatactg

SEQ ID NO: 5 - DNA, 73 bp, Synthetic construct
  source 1..73: mol_type = other DNA
tcgattgtgtggttgtatactgcggcgggcctgcgggagatgcggtggtccttgagaccgctcgaccgaaaaa

SEQ ID NO: 6 - DNA, 46 bp, Synthetic construct
  source 1..46: mol_type = other DNA
tttttcccgagctacaccgaaggtacgaaccgcggctccacaaccg

SEQ ID NO: 7 - DNA, 27 bp, Synthetic construct
  source 1..27: mol_type = other DNA
gacacaatcgatttttcggtcgttttt

SEQ ID NO: 8 - DNA, 29 bp, Synthetic construct
  source 1..29: mol_type = other DNA
attgtgggtcttgaggctcggcccgccgc

SEQ ID NO: 9 - DNA, 35 bp, Synthetic construct
  source 1..35: mol_type = other DNA
cttcattccactccgctccatctctaactcacact

SEQ ID NO: 10 - DNA, 29 bp, Synthetic construct
  source 1..29: mol_type = other DNA
gcggcgggccgagcctcaagacccacaat

SEQ ID NO: 11 - DNA, 19 bp, Synthetic construct
  source 1..19: mol_type = other DNA
gagcctcaagacccacaat

SEQ ID NO: 12 - DNA, 10 bp, Synthetic construct
  source 1..10: mol_type = other DNA
ggcccgccgc

(19) United States
(12) Patent Application Publication
(10) Pub. No.: US 2026/0035736 A1
(43) Pub. Date: Feb. 5, 2026

(54) NOVEL TRANSMEMBRANE SENSORS AND METHOD OF CHARACTERIZATION FOR LYSIS-FREE DETECTION OF INTRACELLULAR TARGETS

(71) Applicant: Arizona Board of Regents on Behalf of Arizona State University, Scottsdale, AZ (US)

(72) Inventors: Rizal F. HARIADI, Phoenix, AZ (US); Nirbhik ACHARYA, Tempe, AZ (US); Carter SWANSON, Richmond, VA (US)

(21) Appl. No.: 19/363,771

(22) Filed: Oct. 21, 2025

Related U.S. Application Data
(63) Continuation of application No. PCT/US24/25661, filed on Apr. 22, 2024.
(60) Provisional application No. 63/497,607, filed on Apr. 21, 2023.

(51) Int. Cl. C12Q 1/6825; C12N 15/113; C12Q 1/6809; C12Q 1/6823

(52) U.S. Cl. CPC C12Q 1/6825; C12N 15/113; C12Q 1/6809; C12Q 1/6823; C12N 2310/3517; C12Q 2600/178

(57) ABSTRACT

The present disclosure provides compositions and methods related to nucleic acid sensors. In particular, the present disclosure provides nucleic acid sensors that can span through lipid bilayer membranes to detect internal nucleic acid targets present in vesicles.

### PRIORITY STATEMENT

This application is a continuation of International Application No. PCT/US2024/025661, filed Apr. 22, 2024, which claims priority to U.S. Provisional Patent Application No. 63/497,607, filed Apr. 21, 2023, the entire contents of which are incorporated herein by reference for all purposes.

### STATEMENT REGARDING FEDERAL FUNDING

This invention was made with government support under AI144247 awarded by the National Institutes of Health. The government has certain rights in the invention.

### SEQUENCE LISTING STATEMENT

The contents of the electronic sequence listing titled (SKYSG-41937-601.xml; Size: 11,934 bytes: and Date of Creation: Apr. 22, 2024) is herein incorporated by reference in its entirety.

### FIELD

The present disclosure provides compositions and methods related to nucleic acid sensors. In particular, the present disclosure provides nucleic acid sensors that can insert through the lipid bilayer and detect internal nucleic acid targets present in vesicles.

### BACKGROUND

Non-invasive detection of nucleic acid targets, such as mRNA and miRNA, in live cells, is an area of intense interest due to its potential for biomedical applications. Technological advancements, such as q-RT-PCR and RNA sequencing, have enabled the elucidation of the functional mechanisms of different RNA species, such as siRNA, miRNA, and IncRNA, and cellular differentiation. However, quantification of nucleic acid levels in living cells poses a significant challenge due to the unavailability of suitable tools. Preceding biological sample processing can be disruptive, and certain rare cell populations with unique transcriptomic profiles may remain unexplored for functional studies if the only distinguishable biomarkers necessitate cell lysis for quantification. Accordingly, there exists a need for new and improved lysis-free methods for detecting nucleic acid intracellularly.

### SUMMARY

The present disclosure provides a DNA sensor that can detect nucleic acid targets enclosed within lipid vesicles using a flip-flop mechanism. In some embodiments, the sensor comprises a hydrophobic tag that facilitate the insertion of the sensor through the lipid bilayer membrane of the vesicle. In some embodiments, the sensor can detect cellular and exosomal nucleic acids within lipid vesicles in a non-invasive manner without breaking cells and exosomes. The provided nucleic acid sensors are useful in applications related to nucleic acid detection technology and provide a valuable tool for detecting and analyzing nucleic acids in biological samples enclosed within lipid membranes.

### BRIEF DESCRIPTION OF THE DRAWINGS

FIG. 1 shows representative schematic diagrams of a nucleic acid sensor according to one embodiment of the present disclosure. The sensor is shown in closed and open format. The sequences provided in the diagrams correspond to potential sequences provided in Table 1.

FIGS. 2A-2D show efficacy of the sensor design. (FIG. 2A) Comparison of the relative fluorescence of the new sensor (200 nM) in the OFF and ON state shows that in solution there is >40 times gain in sensor fluorescence upon hybridisation with equimolar target DNA (200 nM). Error bars are ±SD (N=3). (FIG. 2B) Determination of new sensor's dynamic range for in-solution target sensing. Relative sensor fluorescence (sensor concentration=200 nM) has been plotted as a function of target concentration. Error bars are ±SD (N=3). The plot has been replotted on log scale (inset) which can be fitted to a linear function between 4-200 nM target, suggesting improved sensor's high in-solution dynamic range for target detection. Comparison of the sensor switch on (fluorescence gain) kinetics of the previous (old) sensor design (FIG. 2C) and the new improved sensor design (FIG. 2D) as a function of time.

FIG. 3A-3D show TraNS-lipid membrane interaction, insertion and target detection in GUVs. TraNS-lipid membrane interaction in the absence (FIG. 3A) and presence (FIG. 3B) of cholesterol modification. Panel A (i) and B (i) shows a large field of view: A (ii) and B (ii) are zoomed-in to show single GUV: A (iii) and B (iii) shows representation of cross section view of a GUV with cholesterol unmodified and modified sensors. Halo-ring assay for GUV encapsulated target detection (FIG. 3C (i and ii) and FIG. 3D (i and ii)). Panel (i) and (ii) respectively show confocal images and representative schematics showing: no halo-ring formation for GUV^{Scrambled} (C (i and ii)) and halo-ring formation for GUV^{Target} (FIG. 3D (i and ii)). Panel C (iii) and D (iii) compares the confocal images for the sensor fluorescence for GUVscrambled and GUV^{Target}. The sensor fluorescence intensity distribution has been compared for GUV^{Scrambled} (C (iv)) and GUV^{Target} (D (iv)) from the respective confocal images. All scale bars correspond to 10 μm.

FIGS. 4A-4B show higher TraNS-membrane insertion with membrane crowding. (FIG. 4A) The TraNS-target recruitment in GUVs without membrane crowding (Top) and with membrane crowding (bottom) has been compared by confocal imaging monitoring the halo ring formation from Cy5 tagged target DNA. The scale bar corresponds to 10 μm. (FIG. 4B) The target sensing efficiency of TraNS (% GUVs with target sensing) compared in the absence and presence of membrane crowding by TraNS^{Dark} (TraNS^{Dark} is shown as faint sensors in the representation). N=9 field of views for “No Crowding” and 7 field of views for “Crowding” condition from two sets of experiments.

FIGS. 5A-5D show an examination of GUVs' membrane integrity after target sensing by TraNS. (FIG. 5A) Schematic for Atto488 labeled 21-nt scrambled DNA leakage assay. (FIG. 5B) and (FIG. 5C) show confocal images for the Cy5 labeled target DNA and Atto488 labeled scrambled DNA, respectively. (FIG. 5D) Comparison of fluorescence intensity of Atto488 for GUVs with and without halo-ring formation. The scale bar corresponds to 10 μm.

FIGS. 6A-6B show TraNS-cell interaction and transmembrane insertion through plasma membrane. Schematic and confocal images of TraNS cell membrane interaction monitored at various time points (5 min, 15 min, 30 min, and 60 min) are shown for TraNS^{Cy3-Cy5} (FIG. 6A) and TraNS^{Cy5-Cy3} (FIG. 6B). The green channel corresponds to Cy3 signal, whereas the red channel corresponds to Cy5 channel. All scale bars correspond to 10 μm.

FIGS. 7A-7C show different conformations for a Holliday junction, as described herein. FIG. 7A shows an “open” Holliday junction, FIG. 7B shows a parallel “stacked” Holliday junction, and FIG. 7C shows an anti-parallel “stacked” Holliday junction.

### DETAILED DESCRIPTION

The present disclosure provides a sensor that can insert through the lipid bilayer and detect internal nucleic acid targets present in vesicles. The sensor provided herein is also referred to as a “nucleic acid sensor”, a “transmembrane sensor”, a “transmembrane nanosensor”, or “TraNS”.

The sensor utilizes a novel approach that leverages toehold-mediated strand displacement (TMSD), and DNA hybridization design. The sensor creates a 2-helix DNA nanostructure with sequences, structures, and hydrophobic decoration designed to span through lipid bilayer membranes. The sensor detects internal nucleic acids of interest by generating a fluorescence signal output. The design of the sensor performs exceptionally well in-solution and in detecting targets in cell-sized vesicles.

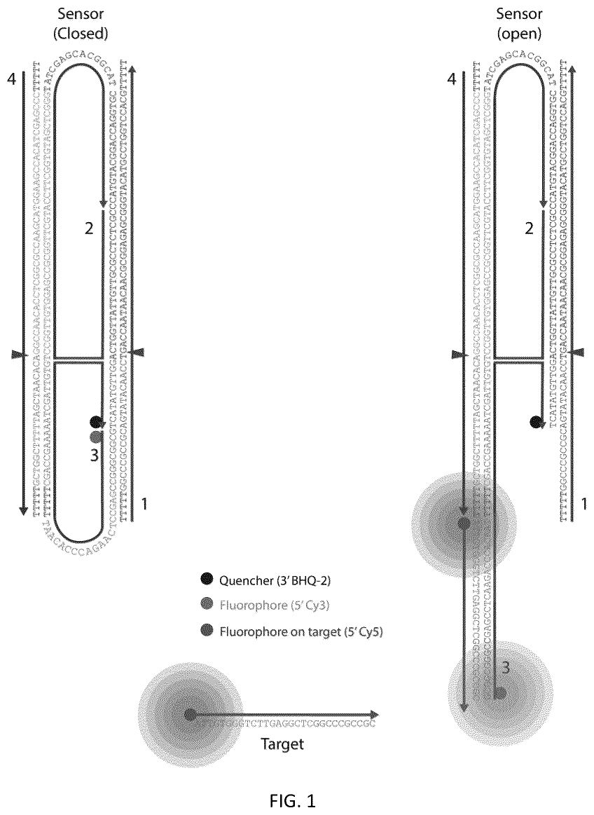

The DNA sensor described herein is a two-helix structure comprising four DNA strands that form an internal Holliday junction and two loops at either end (FIG. 1). One of the loops serves as a toehold for the binding of target nucleic acids. Two of the four strands in the sensor contain an internal hydrophobic tag that is strategically placed to create an asymmetric design. In some embodiments, cholesterol is used as the hydrophobic tag. Other suitable hydrophobic tags can be used, such as alkyl chains or aromatic compounds. This asymmetric design confers an advantage to the sensor by facilitating its insertion through the lipid bilayer by creating enough imbalance for the polar backbone of the sensor to eventually insert through the membrane, allowing for the detection of nucleic acid targets within the cell/vesicle. In some embodiments, the sensor comprises an indicator that produces a detectable shift in signal upon binding of a target nucleic acid to the sensor. In some embodiments, the indicator is present on a portion of the sensor that remains on the extracellular side of the lipid membrane. In other words, in some embodiments the indicator is on an extracellular region of the sensor, after the sensor inserts into the lipid membrane of a cell. Accordingly, a detectable shift in the signal given by the indicator can be detected while still providing information about intracellular targets. The provided nucleic acid sensor is useful in applications related to DNA sensing technology and as a valuable tool for the detection and analysis of nucleic acids in biological samples.

Embodiments of the present disclosure provide nanometer-sized biosensors fabricated from DNA that can span through lipid bilayer membranes, detect internal nucleic acid targets present in vesicles (such as cells and exosomes), and emit a detectable signal from an extracellular portion of the biosensor.

In various embodiments, the present disclosure provides a unique approach to detecting nucleic acid targets enclosed within lipid membranes of biological systems, such as exosomes, cells, and tissues. The sensor of the disclosure can transduce signals across the lipid membrane non-invasively, without compromising membrane integrity or breaking exosomes or cells open. The absence of lysis results in a faster protocol with reduced steps, reduced sample loss, and potentially lower Limit of Detection. The provided sensors and methods have a wide range of applications in various fields, including medical diagnosis and drug development. Other applications include live-cell isolation, exosome diagnostics, cell therapy, disease diagnosis, cell genotyping, and targeted therapeutics.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “single-stranded” oligonucleotides generally refers to those oligonucleotides that contain a single covalently linked series of nucleotide residues.

“Complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, to place the elements in the proper conformation for complementarity, to co-localize complementary elements, to lower interaction energy for complementation, etc.

When used in reference to polynucleotides (e.g., a sequence of nucleotides such as all or a portion of a nucleic acid molecule or a target nucleic acid), the terms “complementary” or “complementarity” are used in reference to polynucleotides related by the base-pairing rules. For example, the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary,” “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. As an example, nucleotides that can form base pairs, e.g., that are complementary to one another, include the pairs: cytosine and guanine, thymine and adenine, and adenine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100,or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

The term “domain of complementarity” as used herein refers to a region of one structural element (e.g., nucleic acid molecule) that is complementary relative to a specified other structural element (e.g., target nucleic acid).

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure comprises a “double-stranded nucleic acid”. For example, triplex structures are considered to be “double-stranded.” In some embodiments, any base-paired nucleic acid is a “double-stranded nucleic acid.”

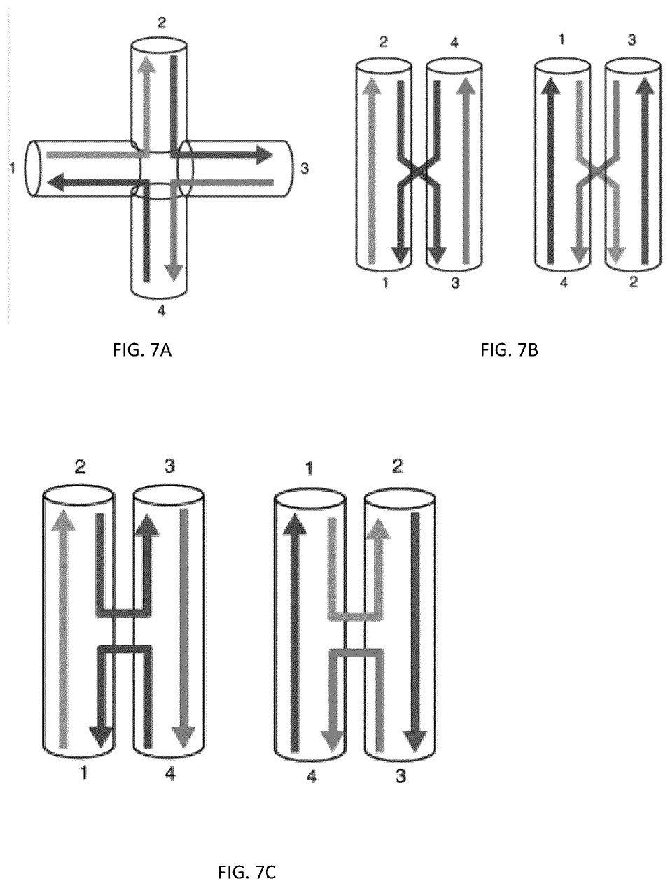

The term “Holliday junction” refers to a branched nucleic acid structure where four different nucleic acid strands intersect. A “Holliday junction” is thus considered a four-way DNA structure. The exact structure of a Holliday junction can differ depending on binding conditions. A Holliday junction can take on an “open” conformation where electrostatic repulsion keeps the junction in an unstacked position with the four arms directed towards the corners of a square. This type of Holliday junction comprises four double-stranded arms, resembling a cross, as shown in FIG. 7A. Alternatively, a Holliday junction can take on a “stacked” conformation, where with two of the four strands running parallel or anti-parallel to each other and the other two strands “stacking” atop one another. A stacked Holliday junction can be parallel (i.e. strands 1 and 2 run parallel to each other) or anti-parallel (i.e. strands 1 and 2 run antiparallel to each other). A “stacked” Holliday junction formation is shown in FIG. 1.

Specifically, FIG. 1 shows an antiparallel stacked-X Holliday junction. Note that the “open” configuration of the sensor after binding of the target and strand displacement is not the same as an “open” conformation of a Holliday junction described above. Additionally exemplary configurations for a “stacked” parallel Holliday junction is shown in FIG. 7B, and exemplary configurations for a “stacked” anti-parallel Holliday junction is shown in FIG. 7C.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the terms “nucleotide sequence identity” or “nucleic acid sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 5, 10, 15, 20, 25, 30, 35, 40, 45, 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences: (b) dividing the number of matched positions by the total number of positions in the window of comparison: and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981: Needleman & Wunsch, 1970); Pearson & Lipman, 1988: Ausubel et al., 1988; and Sambrook & Russell, 2001.

As used herein, “hybridization chain reaction (HCR)” is an enzyme-free isothermal nucleic acid amplifying process. It involves using two or more metastable monomer hairpins that react with an initiator strand to initiate the polymerization process (Dirks and Pierce, 2004: US Patent US20050260635A1: US Patent US20120190835A1). To trigger the polymerization of the monomers, the initiator strand is introduced. The potential for programmability of HCR has been utilized in numerous applications, including the detection of DNA and RNA, as well as RNA imaging in fixed cells (Choi et al, 2010; Molecular Instruments Inc.).

As used herein, “toehold-mediated strand displacement (TMSD)” refers to a process in which a DNA strand in a DNA helix structure called the protector strand can be displaced and replaced by an invader strand that is complementary to the other strand in the original helix structure. The other strand in the original helix structure is called the original strand, which has a region (e.g. an overhang) called a “toehold” that assists the invading strand in dislodging and replacing the protector strand. Yurke et al introduced the concept of TMSD to the field of DNA nanotechnology where they constructed a nanomolecular machine powered by DNA (Yurke et al. Nature., 2000, Vol.406 (6796), p.605-608). The TMSD process has many applications such as DNA nanotechnology, DNA molecular machines, DNA computing, DNA sensing, and programmable DNA nanostructures, among others.

The term “toehold domain” as used herein refers to a region of a nucleic acid molecule that can function as a toehold (e.g. a region, such as an overhang, that assists the invading strand in dislodging and replacing the protector strand) in a TMSD process. In some embodiments, the toehold domain is complementary relative to at least a portion of a specified other nucleic acid molecule (e.g., a target nucleic acid). In some embodiments, the toehold domain is complementary to a target nucleic acid.

### 2. Nucleic Acid Sensors

Embodiments of the present disclosure include compositions and methods related to nucleic acid sensors. In particular, the present disclosure provides nucleic acid sensors that can span through lipid bilayer membranes to detect internal nucleic acid targets. In various embodiments, these sensors provide for lysis-free detection of internal nucleic acids (such as mRNA) in vesicles, including exosomal and live-cell nucleic acids.

In various embodiments, a nucleic acid sensor of the present disclosure is a two-helix (2-helix) structure comprising four DNA strands that form an internal Holliday junction and two loops at either end (FIG. 1). In some embodiments, the nucleic acid sensor comprises four separate nucleic acid strands that form a Holliday junction with four double-stranded arms joined at the junction and two loops positioned at opposing ends of the sensor. For example, this type of Holliday junction can be formed when electrostatic repulsion forces are sufficiently high, pushing the strands into a fully open Holliday junction resembling a cross. In some embodiments, the nucleic acid strand comprises four separate nucleic acid strands that form a stacked Holliday junction with two of the four nucleic acid strands joined at the junction, the other two arms running either parallel or anti-parallel to each other, and two loops positioned at opposing ends of the sensor. For example, as shown in FIG. 1 in some embodiments the nucleic acid sensor forms this type of “stacked” Holliday junction conformation. In FIG. 1. strands 1 and 4 (S1 and S4) run antiparallel to each other and portions of strands 2 and 3 (S2 and S3) intersect, thereby forming a stacked Holliday junction. The portions of S2 and S3 that intersect are shown in FIG. 1 as the parallel gray lines in cross proximity to each other. In other suitable configurations, two strands (e.g. S1 and S4) can run parallel to each other and portions of the other two strands (e.g. S2 and S3) intersect to form the stacked Holliday junction. In some embodiments, at least one of the two loops comprises a toehold domain complementary to a target nucleic acid. In some embodiments, at least one of the two loops comprises a toehold domain complementary to a target nucleic acid, two of the four separate nucleic acid strands comprise an internal hydrophobic tag, and one nucleic acid strand comprises an indicator that produces a detectable shift in signal upon binding of a target nucleic acid to the nucleic acid sensor. For example, in some embodiments upon binding of a target nucleic acid to the toehold domain of a loop induces strand displacement resulting in a detectable shift in signal from the indicator.

In some embodiments, two of the four separate nucleic acid strands comprise an internal hydrophobic tag. In some embodiments, each hydrophobic tag is placed asymmetrically within the sensor. The hydrophobic tags being placed asymmetrically within the sensor indicates that the position of the hydrophobic tag is not in the middle of the sensor, which thus divides the sensor into a longer and a shorter segment. Without wishing to be bound to any one particular theory, it is thought that this asymmetric design confers an advantage to the sensor by facilitating imbalance for the polar backbone of the sensor to eventually flip across the membrane. In some embodiments, each hydrophobic tag is placed symmetrically within the sensor.

In some embodiments, each hydrophobic tag is cholesterol. In some embodiments, one or two of the hydrophobic tags is a moiety other than cholesterol. Suitable hydrophobic moieties include, for example, lipid molecules, alkyl chains or aromatic compounds. Approaches for attaching lipid molecules, such as cholesterol, to DNA phosphate backbones are known in the art. For example, some approaches use triethylene glycol (TEG) links. In some embodiments, the first nucleic acid strand (e.g. Strand 1 (S1) and the fourth nucleic acid strand (e.g. Strand 4 (S4)) each contain a hydrophobic tag (e.g. cholesterol). In FIG. 1, exemplary locations for the hydrophobic tags on S1 and S4 are shown by the triangles. These locations are only intended to be exemplary, the hydrophobic tags can be placed at any suitable location to achieve asymmetrical or symmetrical placement of the tags.

In some embodiments, at least one of the two loops comprises a toehold domain complementary to a target nucleic acid. In some embodiments, only one of the two loops comprises a toehold domain complementary to a target nucleic acid. In some embodiments, each of the two loops comprises a toehold domain complementary to a target nucleic acid. In some embodiments, the toehold domain consists of 13-19 nucleotides that are complementary to the target nucleic acid. The sequence of the toehold domain will depend on the target nucleic acid to be detected.

In some embodiments, a portion of a given strand of the sensor forms a loop comprising a toehold domain. In FIG. 1, a portion of strand 3 forms a loop comprising a toehold domain. However, it is understood that a different strand (e.g. strand 2) may additionally or alternatively form a loop comprising a toehold domain. In the stacked Holliday junction conformation, at least one of the two strands that intersects comprises a loop comprising a toehold domain. In some embodiments, the strand that forms the loop comprising the toehold domain additionally comprises an indicator. In some embodiments, binding of the target nucleic acid to the toehold domain of a given strand displaces a portion of the given strand from the complementary portion of another strand within the sensor, thereby producing a detectable shift in signal of the indicator due to the movement of the indicator that occurs during this displacement.

In this exemplary representation shown in FIG. 1, the target sequence is 5′-ATTGTGGGTCTTGAGGCTCGGCCCGCCGC-3 (SEQ ID NO: 8). Bold font indicates the portion of the target sequence that is complementary to the toehold domain. The sensor is designed such that a given strand forms a loop comprising a toehold domain that is complementary to a portion of the target sequence. Specifically, the toehold domain as shown in FIG. 1 comprises the sequence GAGCCTCAAGACCCACAAT (SEQ ID NO: 11), which is complementary to the portion of the target sequence shown in bold above. The remaining portion of the target sequence not in bold text (GGCCCGCCGC. SEQ ID NO: 12) is complementary to another portion of the given strand (e.g. strand 3 in FIG. 1) adjacent to (e.g. upstream of) the toehold domain. Binding of the target sequence to the toehold domain displaces this portion of the given strand (e.g. the portion of strand 3 adjacent to the toehold domain in FIG. 1) from the complementary portion of another strand in the sensor (e.g. strand 1 in FIG. 1). In some embodiments, this displacement results in a detectable shift in signal from the indicator component by physically spacing the indicator component (e.g. fluorophore) from a quencher.

In some embodiments, one nucleic acid strand comprises an indicator that produces a detectable shift in signal upon binding of the target nucleic acid to the nucleic acid sensor. In some embodiments, the indicator comprises a fluorophore. Any suitable fluorophore may be used. In some embodiments, one nucleic acid strand comprises the fluorophore and one nucleic acid strand comprises a quencher, wherein the fluorophore and quencher are positioned such that a signal from the fluorophore is quenched in the absence of the target nucleic acid. For example, in the absence of the target nucleic acid the nucleic acid the fluorophore and the quencher are positioned suitably close to one another, either on the same strand or on separate strands, such that the signal from the fluorophore is quenched. In some embodiments, binding of the target nucleic acid to the nucleic acid sensor results in strand displacement which releases the fluorophore from the quencher, thus producing the detectable shift in signal. For example, in some embodiments binding of the target nucleic acid to the toehold domain displaces a portion of the strand from the complementary portion of a different strand in the sensor. This displacement physically spaces the fluorophore from the quencher. In some embodiments, the fluorophore and the quencher are on the same nucleic acid strand. In some embodiments, the fluorophore and the quencher are on different nucleic acid strands. In some embodiments, the fluorophore is on the third nucleic acid strand. In some embodiments, the fluorophore and the quencher are on the third nucleic acid strand.

In some embodiments, binding of the target nucleic acid to the toehold domain results in toehold-mediated strand displacement (TMSD), removing the quencher from the fluorophore and thus permitting a detectable signal to occur from the fluorophore. For example, as shown in FIG. 1 in some embodiments the fluorophore and the quencher are each on the nucleic acid strand comprising the toehold domain (e.g. strand 3). In this embodiment the fluorophore and the quencher are placed on the same strand proximal to one another such that the signal is quenched in the absence of the target nucleic acid. Upon binding of the target strand to the toehold domain, a portion of the strand (e.g. strand 3) adjacent to the toehold domain is displaced from the complementary portion of a different strand (e.g. strand 1), physically distancing the fluorophore from the quencher such that a detectable signal is produced. In this embodiment, the location of the fluorophore and the quencher is selected such that one entity is placed at a position of the strand (e.g. strand 3) that is complementary to the portion of the target strand, such that displacement results in binding of this portion of the strand (e.g. strand 3) to the target strand, whereas the other entity is placed at a position of the strand (e.g. strand 3) that is complementary to another strand on the sensor (e.g. strand 1) and not complementary to the target sequence, such that binding to the other strand of the sensor is not displaced by binding of the target sequence to the toehold domain. This is shown visually in FIG. 1, where the fluorophore is placed on a position of strand 3 which binding to strand 1 is disrupted upon binding of the target sequence to the toehold domain, whereas the quencher is placed at a position of strand 3 that remains undisrupted (e.g. bound to the complementary portion of strand 1) following binding of the target sequence to the toehold domain of the sensor. The opposite arrangement of the fluorophore and the quencher may also be used, where the quencher is removed from the fluorophore by displacement.

A similar arrangement can be used wherein the fluorophore and the quencher are on two separate strands. For example, the fluorophore can be placed in a suitable position on strand 3, as shown in FIG. 1, and the quencher can be placed at a location on strand 1 that is sufficiently proximal to the position of the fluorophore such that in the absence of the target strand, the fluorophore is quenched. Upon finding of the target sequence to the toehold domain, the fluorophore is removed from the quencher by displacement, resulting in a detectable signal. As with the above, the opposite arrangement of the fluorophore and the quencher may also be used, where the quencher is removed from the fluorophore by displacement.

In some embodiments, the fluorophore is positioned on an extracellular region of the sensor, such that the signal from the fluorophore corresponding to an intracellular binding event is able to be detected extracellularly.

In some embodiments, each of the four different strands of the sensor is from about 30) to about 100 nucleotides. In some embodiments, each of the four different strands of the sensor is from about 25 to about 50 nucleotides. In some embodiments, each of the four different strands of the sensor is from about 35 to about 100 nucleotides. In some embodiments, each of the four different strands of the sensor is from about 45 to about 50 nucleotides. In some embodiments, each of the four different strands of the sensor is from about 15 to about 40 nucleotides. In some embodiments, each of the four different strands of the sensor is from about 15 to about 30 nucleotides. In some embodiments, each of the four different strands of the sensor is from about 15 to about 20 nucleotides. All four of the different strands of the sensor do not need to be of the same length of nucleotides. In some embodiments, two of the four different strands of the sensor are characterized by a first length and the remaining two of the four arms are characterized by a second length.

In some embodiments, the nucleic acid sensor is a DNA molecule, a locked nucleic acid (LNA) molecule, or combinations thereof. In some embodiments, the nucleic acid sensor is an RNA molecule.

In some embodiments, the target nucleic acid is a DNA molecule or an RNA molecule. In some embodiments, the target nucleic acid is an mRNA, miRNA, or IncRNA molecule.

Embodiments of the present disclosure also include a composition comprising any of the nucleic acid sensors described herein. In some embodiments, the composition comprises at least target nucleic acid.

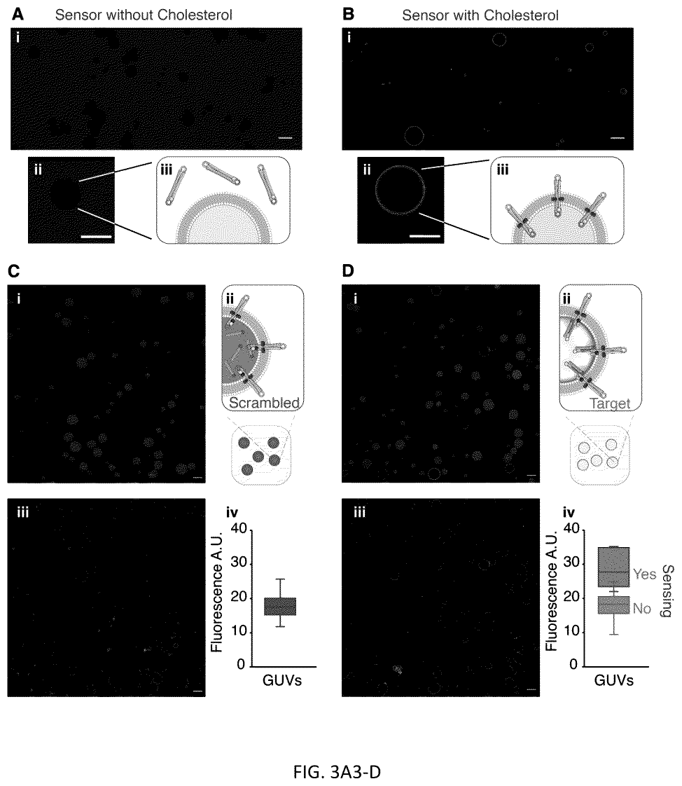

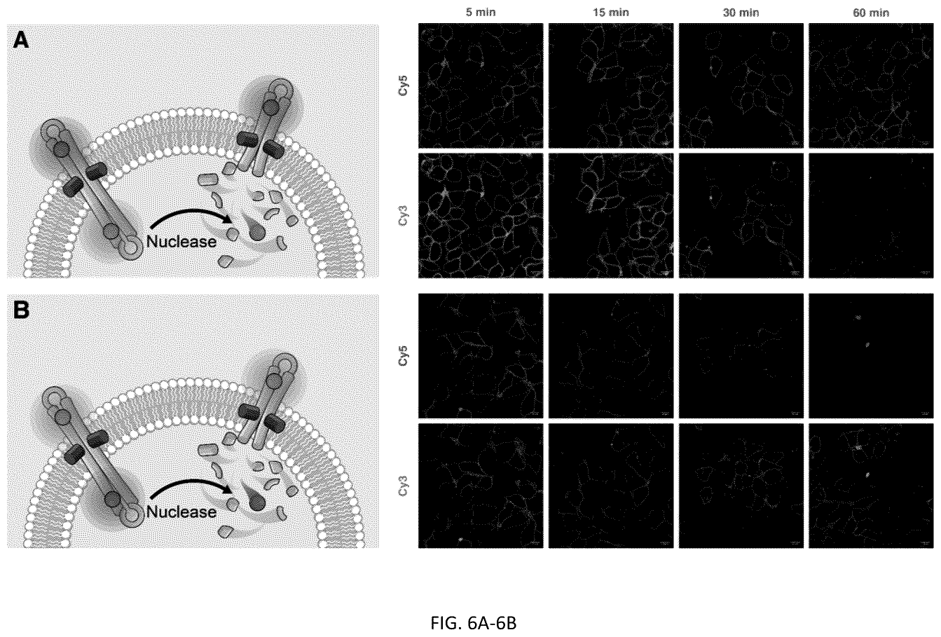

Embodiments of the present disclosure also include a method of detecting a target nucleic acid using any of the sensors or compositions described herein. In some embodiments, the target nucleic acid is located within a membranous vesicle or cell. A nucleic acid located within a membrane (e.g. within a membranous vesicle or within a cell) is referred to herein as “intracellular”. In some embodiments, the method comprises detecting the target nucleic acid without lysis of the membranous vesicle or cell. In some embodiments, the target nucleic acid is DNA or RNA. In some embodiments, the method comprises contacting a sample with a sensor provided herein, and detecting a signal from the sensor. In some embodiments, a detectable change in signal from the sensor (e.g. an increased signal from the sensor resulting from displacement of the fluorophore from the quencher) indicates that the target is present in the sample. In some embodiments, the sample comprises a membranous vesicle or cell and the presence or absence of the target is detected without lysing the membranous vesicle or cell. In some embodiments, during use the sensor adopts a transmembrane configuration in which one portion of the sensor is inserted across the cell membrane into an intracellular location and wherein a different portion of the sensor is exposed at an extracellular location. For example, in some embodiments the portion of the sensor containing the toehold domain complementary to the target nucleic acid is inserted across the cell membrane into the intracellular location and a portion of the sensor containing the indicator is exposed at the extracellular location. Such a sensor enables detection of a target by visualizing or quantifying the signal extracellularly. Such a sensor is shown in FIG. 6. As another example, in some embodiments a portion of the sensor containing the toehold domain complementary to the target nucleic acid and a portion of the sensor containing the indicator is inserted across the cell membrane into the intracellular location, and a portion of the sensor containing the separate loop not containing the toehold domain is exposed at the extracellular location. In such embodiments the target nucleic acid is detected by visualizing or quantifying the change in signal intracellularly. Such a sensor is shown in FIG. 3D.

In some embodiments, a method of detecting a target nucleic acid comprises use of a neutralizing nucleic acid that is applied externally to the vesicle or cell. In some embodiments, the neutralizing nucleic acid comprises a sequence that is complementary to at least a portion of the target nucleic acid.

In some embodiments, the method comprises a target detection step. For example, in some embodiments, nucleic acid detection includes the use of a fluorophore, a chromophore, fluorophore pairs, fluorophore-quencher pairs or other detection moiety known in the art.

Embodiments of the present disclosure also include a kit comprising any of the sensors described herein, and instructions for detecting a target nucleic acid. In some embodiments, the kit further comprises at least one reporter nucleic acid, wherein the at least one reporter nucleic acid comprises a sequence that is complementary to at least a portion of a nucleic acid molecule of the sensor. In some embodiments, the kit further comprises a neutralizing nucleic acid, wherein the neutralizing nucleic acid comprises a sequence that is complementary to at least a portion of the target nucleic acid.

### 3. Sequences

The various nucleic acid sequences referenced herein are provided below. In Table 1. “/3Cy5Sp/” and “/3Cy3Sp/” refer to fluorophores and “/iCholTEG/” refers to a hydrophobic tag.

TABLE 1
DNA strand sequences for TraNS.

| Sensor strands | Sequence |
|---|---|
| Strand 1 (S1) | TTT TTG GCC CGC CGC AGT ATA CAA CCT |
| | (SEQ ID NO: 1) /iCholTEG/GAC CAA TAA CAA |
| | CGC GGA GAG CGG GTA CAT GCC TGG TCC |
| | ACG TTT TT (SEQ ID NO: 2) |
| Strand 2 (S2) | CGC TCT CCG CGT TGT TAT TGG TCA CCG GTT |
| | GTG GAG CCG CGG TTC GTA CCT TCG GTG |
| | TAG CTC GGG TAT CGA GCA CGG CAT CGT |
| | GGA CCA GGC ATG TAC C (SEQ ID NO: 3) |
| Strand 3 (S3) | /5Cy3/CGG CGG GCC GAG CCT CAA GAC CCA |
| | CAA TTT TTT CGA CCG AAA AAT CGA TTG |
| | TGT GGT TGT ATA CTG (SEQ ID NO: 4)/3IABKFQ/ |
| Strand 3 non-sense | TCGATTGTGTGGTTGTATACTGCGGCGGGCCTG |
| | CGGGAGATGCGGTGGTCCTTGAGACCGCTCGA |
| | CCGAAAAA (SEQ ID NO: 5) |
| Strand 4 (S4) | TTT TTC CCG AGC TAC ACC GAA GGT ACG |
| | AAC CGC GGC TCC ACA ACC G (SEQ ID NO: |
| | 6)/iCholTEG/GA CAC AAT CGA TTT TTC GGT |
| | CGT TTT T (SEQ ID NO: 7) |
| Target strand | /5Cy5/ATT GTG GGT CTT GAG GCT CGG CCC |
| | GCC GC (SEQ ID NO: 8) |
| Scrambled strand | /5Cy5/CTT CAT TCC ACT CCG CTC CAT CTC |
| | TAA CTC ACA CT (SEQ ID NO: 9) |
| Target complementary | GCG GCG GGC CGA GCC TCA AGA CCC ACA AT |
| strand | (SEQ ID NO: 10) |
| Strand 2 Cy3 | CGC TCT CCG CGT TGT TAT TGG TCA CCG GTT |
| | GTG GAG CCG CGG TTC GTA CCT TCG GTG |
| | TAG CTC GGG TAT CGA GCA CGG CAT CGT |
| | GGA CCA GGC ATG TAC C (SEQ ID NO: |
| | 3)/3Cy3 Sp/ |
| Strand 3 Cy5 | CGG CGG GCC GAG CCT CAA GAC CCA CAA |
| | TTT TTT CGA CCG AAA AAT CGA TTG TGT GGT |
| | TGT ATA CTG (SEQ ID NO: 4) /3Cy5Sp/ |
| Strand 2 Cy5 | CGC TCT CCG CGT TGT TAT TGG TCA CCG GTT |
| | GTG GAG CCG CGG TTC GTA CCT TCG GTG |
| | TAG CTC GGG TAT CGA GCA CGG CAT CGT |
| | GGA CCA GGC ATG TAC C (SEQ ID NO: |
| | 3)/3Cy5Sp/ |
| Strand 3 Cy3 | CGG CGG GCC GAG CCT CAA GAC CCA CAA |
| | TTT TTT CGA CCG AAA AAT CGA TTG TGT GGT |
| | TGT ATA CTG (SEQ ID NO: 4) /3Cy3Sp/ |

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Sensor Design, Structure and Working Principle.

The present invention relates to a DNA sensor designed to detect nucleic acid targets within vesicles or cells. The DNA sensor is also referred to as a transmembrane sensor, or a transmembrane nanosensor (TraNS). The DNA sensor described herein is a two-helix structure comprising four DNA strands that form an internal Holliday junction (e.g. a stacked Holliday junction) and two loops at either end (FIG. 1). One of the loops serves as a toehold for the binding of target nucleic acids. Two of the four strands in the sensor contain an internal hydrophobic tag that is strategically placed to create an asymmetric design. In some embodiments, cholesterol is used as the hydrophobic tag. Other suitable hydrophobic tags can be used, such as alkyl chains, aromatic compounds. This asymmetric design confers an advantage to the sensor by facilitating its insertion through the lipid bilayer by creating enough imbalance for the polar backbone of the sensor to eventually flip and insert through the membrane. In alternative embodiments a symmetrical design may be used. The present sensors represents a significant improvement in sensor stability and target sensing output.

The sensor comprises two toeholds, each consisting of 13-19 nucleotides. In the design shown in FIG. 1, only one of the toeholds (toehold on strand 3: hereafter denoted as the sensing toehold) is complementary to a part of the target nucleic acid, the other toehold (on strand 2) has a essentially random (not unique) DNA sequence. However, sensors can be modified/designed to have sensing toeholds at both ends of the sensor. The strand 3 of the sensor is tagged with a fluorophore and a quencher on 5′ and 3′ ends, respectively. Upon correct self-assembly of the sensor, the 5′ and 3′ ends of strand 3 come next to each other resulting in the fluorescence quenching of 5′ fluorophore by the 3′ quencher. To provide stability, sensors are designed to be at least 42 bp long. The exemplary sensor shown in FIG. 1 is 63 bp long. Apart of the sensing domains, the sequences are decoupled from the target DNA and can be easily optimized using computational design tools for DNA nanodevices.

Upon insertion into the vesicle, the sensor target strand hybridizes with the sensing toehold via TMSD. Target binding initiated TMSD displaces the 5′ end of strand 3 from the sensor, resulting in an increase in distance between the fluorophore and the quencher. This sensor-target hybridization can be quantified by monitoring the increase in sensor fluorescence using a fluorescence plate reader (for in-solution detection), confocal microscopy (for cells and cell-sized vesicles), and flow cytometry (For cells, and small and cell sized vesicles).

### Exemplary Sensors

Potential sequences for the various strands (4 strands) of the sensors are provided in Table 1. Table 2 highlights different versions of sensors that can be created and/or are used in the examples herein.

TABLE 2
Different versions of sensors and strands
required for their synthesis.

| Name of structure | Required strands |
|---|---|
| Cholesterol untagged | Strand 1, Strand 2, Strand 3, Strand 4 |
| TraNS | |
| Cholesterol tagged | Strand 1-Chol, Strand 2, Strand 3, Strand 4-Chol |
| TraNS | |
| TraNS^{Dark} | Strand 1-Chol, Strand 2, Strand 3 nonsense, |
| | Strand 4-Chol |
| TraNS^{Cy3-Cy5} | Strand 1-Chol, Strand 2 Cy3, Strand 3 Cy5, |
| | Strand 4-Chol |
| TraNS^{Cy5-Cy3} | Strand 1-Chol, Strand 2 Cy5, Strand 3 Cy3, |
| | Strand 4-Chol |

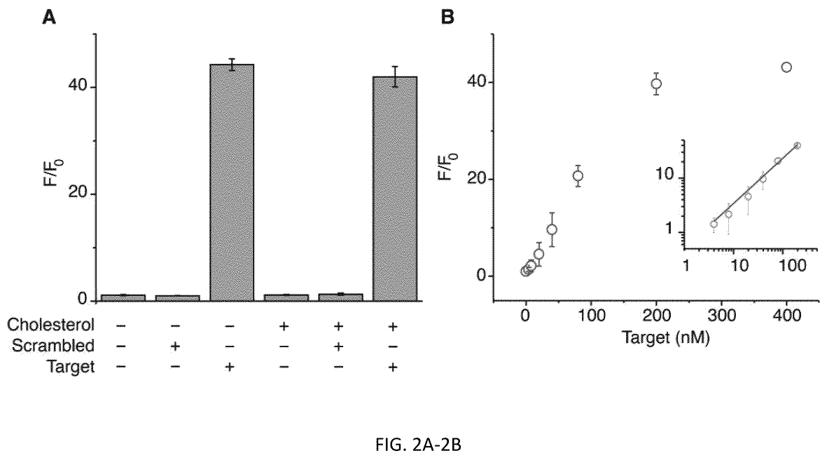

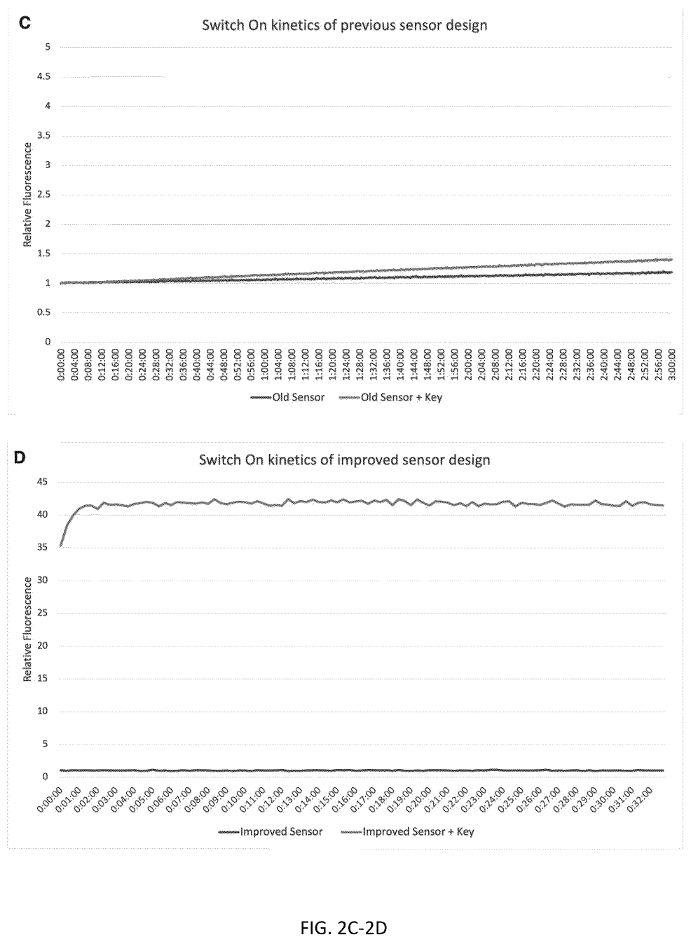

To assess the sensor's target sensing ability in-solution, the fluorescence gain of the sensor upon target binding was measured using a fluorescence plate reader (FIG. 2). The sensor's steady-state fluorescence was monitored in both the OFF state (without target or with scrambled DNA) and the ON state (with equimolar target DNA). The fluorescence of the sensor increased over 40 times upon target sensing in the ON state compared to the OFF state. The fluorescence gain of the sensor was compared to a previous sensor design disclosed in W02022/081766A1 for sensor performance (FIG. 2C and D). The in-solution turn-ON kinetics of the sensor in the presence of equimolar target DNA was measured by monitoring the sensor fluorescence over time on a microplate reader (FIG. 2D). All sensors turned ON within 2 minutes at equimolar concentration of the sensor and target, while the sensor incubated in the folding buffer or with scrambled DNA did not show any fluorescence gain over time. The detection kinetics for the previous design is comparatively very slow (FIG. 2C). The in-solution dynamic range of the sensor for target sensing was tested by performing sensor-target sensing at varying concentrations of target DNA. TraNS shows significant performance for in-solution target detection, with a linear dependency between 2%-100% target availability (FIG. 2B).

### DNA Sensor Insertion Across Lipid Membrane and Target Detection of Single Stranded DNA/Oligonucleotide Within a GUV as a Proof of Concept for Vesicular Target Detection

Proof-of-concept experiments were conducted using cell-sized giant unilamellar vesicles (GUVs) made from phospholipids and cholesterol, encapsulated with target DNA strands. Using fluorescence confocal microscopy, sensor recruitmen and target detection/hybridization were monitored. The results demonstrate the ability of the sensor to detect vesicular target oligonucleotide strands across lipid bilayers.

The giant unilamellar vesicles (GUVs) were prepared by mixing 70% 1-palmitoyl-2-oleoyl-sn-glycero-3-phosphocholine (POPC) and 30% cholesterol, and the inner solution was supplemented with sucrose of a specific concentration and 4 mM Mg2+. To neutralize any leaked target strand in the outer solution, an excess of target complementary strand (fully complementary strand to the target DNA strand) was added in a >20 times molar excess of the target strand and incubated for more than 30) minutes in the test solution. The sensors were mixed with the test solution and incubated for at least 30 minutes before imaging with a confocal microscope. Negative controls included empty GUVs encapsulated with only sucrose solution or scrambled GUVs encapsulated with scrambled DNA. Confocal images of the sensor binding/insertion in GUVs are presented in FIG. 3A and B; the membrane of GUVs with cholesterol-modified sensors appears green due to fluorophore tagging (FIG. 3B), while there is no sensor-membrane binding for cholesterol-untagged sensors (FIG. 3A). To visualize target detection, a ‘Halo ring’ assay was developed, in which the fluorescence of Cy5-labeled oligonucleotides (scrambled/target) encapsulated in GUVs was monitored for recruitment on vesicle membrane via sensor binding. GUVs encapsulating Cy5-labeled scrambled DNA strands showed an evenly dispersed red signal (FIG. 3C (i) and (ii)), while GUVs encapsulating Cy5-labeled target DNA resulted in a red halo ring formation from the fluorophore on the target strand, as it was recruited to the luminal side of the lipid membrane by the sensors (FIG. 3D (i) and (ii)). These results demonstrated the effectiveness of the DNA sensor in detecting nucleic acid targets within vesicles or cells. In addition, fluorescence gain due to target binding was monitored in both GUVs (GUV^{Scrambled} and GUV^{Target}), even though the sensors had an adjacent quencher, and some basal fluorescence from the sensor was observed due to its localization on the membrane. However, the observed sensor fluorescence was very low in scrambled DNA GUVs (FIG. 3C iii and iv). In the test sample (GUVTarget), the sensor fluorescence increased (˜2 times) due to target detection (FIG. 3D iii and iv).

### Membrane Crowding can Increase Sensor-Membrane Insertion Efficiency

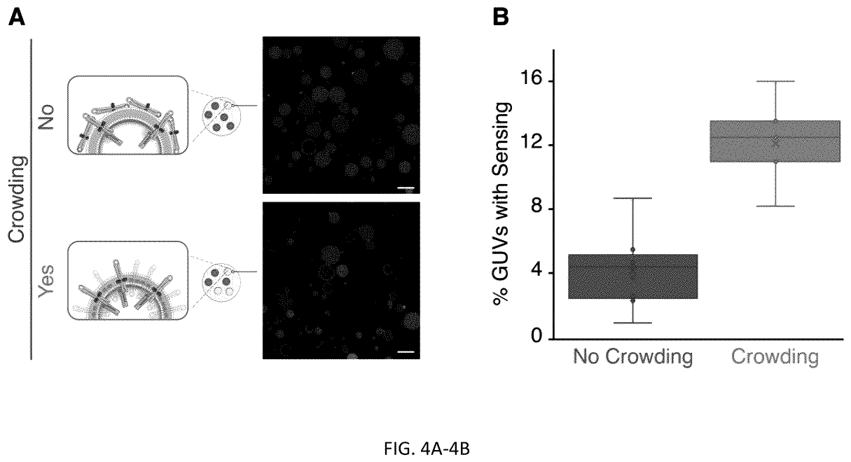

The number of GUVs showing halo-ring formation is low (approximately 4%) (FIG. 3D and FIG. 4). Without wishing to be bound by theory, it is possible that this low efficiency may be attributed to poor insertion, and that crowding of sensors on the bilayer may have a cooperative effect. In order to improve insertion efficiency without compromising the dynamic range, a non-sensing dark sensor was introduced in addition to the TraNS sensor. Inclusion of non-sensing dark sensor (TraNS^{Dark}) resulted in an increase from ˜4% to 12% of GUVs exhibiting halo ring formation (FIG. 4A and B). This effect may be due to steric clashes between sensors in a cis-state on the outer surface of the membrane, which favors adoption of a transmembrane state, leading to a cooperative effect of membrane crowding.

### DNA Nanosensors are Non-Invasive to the GUV Membrane

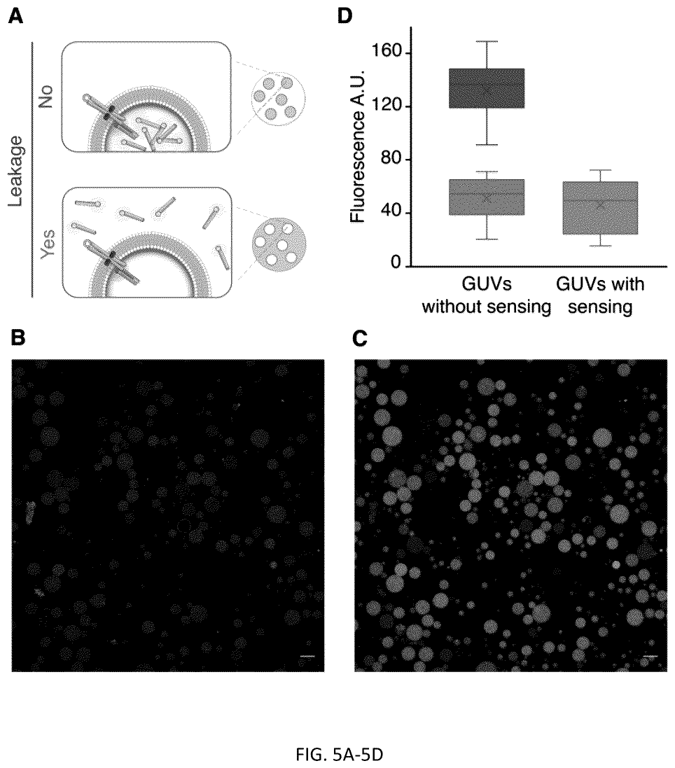

It was next investigated whether transmembrane confirmation of cholesterol modified TraNS affects the lipid bilayer's integrity to retain macromolecules like DNA strands. The leakage of Atto 488 labeled 21-nucleotide scrambled DNA, along with an oligonucleotide target, loaded into the luminal compartment of GUVs before TraNS treatment was monitored. The fluorescence signal of Atto 488 in GUVs with and without halo-ring formation was compared (FIG. 5). In GUVs without halo-ring, the fluorescence signal of Atto 488 showed two distinct populations, one with low fluorescence and another with high fluorescence intensity. This variation may be due to two populations of GUVs formed during preparation with different inner content concentrations or a low number of sensor insertions resulting in partial leakage of GUV contents without halo-ring formation. However, the fluorescence intensity of Atto 488 from all GUVs with target detection was similar to the low fluorescence population of GUVs without target detection (FIG. 5D). If TraNS insertion caused leakage of GUV contents, a significant decrease in the fluorescence signal of Atto 488 from GUVs with target detection would be expected compared to the low fluorescence population of GUVs without target detection. These results suggest that systemic TraNS insertion does not cause significant leakage of small oligonucleotides from GUVs.

### DNA Nanosensor Insert Across Biological Membrane in a Preferred Orientation

The ability of TraNS sensors to interact with human cells (FIG. 6) and adopt a transmembrane conformation on the plasma membrane was next investigated. To test whether DNA segments of the sensor inserted into the cytoplasm would be degraded by nucleases, while extracellular regions would remain intact, two modified variants of TraNS sensors were designed, TraNS^{Cy3-Cy5} and TraNS^{Cy5-Cy3}, in which Strand 2 and Strand 3 were tagged with Cy3and Cy5, respectively. TraNS^{Cy3-Cy5} has Cy3 flagged on the longer segment and Cy5 flagged on the shorter segment, while TraNS^{Cy5-Cy3} has the fluorophore locations swapped.

HEK293T cells were incubated with the sensors for 15 min, and the excess sensors were washed away with fresh live cell imaging solution. Upon incubation with TraNS^{Cy3-Cy5} the Cy3 signal on the cell membrane gradually disappeared within 60 min, while the Cy5 signal remained intact. In contrast, when cells were incubated with TraNS^{Cy5-Cy3}, the Cy5 signal attenuates while the Cy3 signal persists. These data indicate that TraNS inserts through the cell membrane and primarily adopts an orientation in which the longer segment of the sensor is localized on the cytoplasmic side.

### EMBODIMENTS

The present disclosure is further illustrated by the following embodiments:

Embodiment 1: A nucleic acid sensor comprising:
four separate nucleic acid strands, wherein the four separate nucleic acid strands form a Holliday junction and wherein portions of two of the four separate strands form two separate loops positioned at opposing ends of the sensor, wherein,
at least one of the two loops comprises a toehold domain complementary to a target nucleic acid:
two of the four separate nucleic acid strands comprise a hydrophobic tag: and one nucleic acid strand comprises an indicator that produces a detectable shift in signal upon binding of a target nucleic acid to the nucleic acid sensor.

Embodiment 2: The nucleic acid sensor of embodiment 1, wherein the Holliday junction is a stacked Holliday junction wherein the two strands that form two separate loops intersect at the stacked Holiday junction and wherein the other two strands run parallel or antiparallel to each other.

Embodiment 3: The nucleic acid sensor of embodiment 1 or embodiment 2, wherein each hydrophobic tag is placed asymmetrically within the nucleic acid sensor.

Embodiment 4: The nucleic acid sensor of any one of embodiments 1-3, wherein each hydrophobic tag is cholesterol.

Embodiment 5: The nucleic acid sensor of any one of embodiments 1-4, wherein the indicator comprises a fluorophore.

Embodiment 6: The nucleic acid sensor of embodiment 5, wherein one nucleic acid strand comprises the fluorophore and one nucleic acid strand comprises a quencher, wherein the fluorophore and quencher are positioned such that a signal from the fluorophore is quenched in the absence of the target nucleic acid, and wherein binding of the target nucleic acid to the nucleic acid sensor results in strand displacement which releases the fluorophore from the quencher, thus producing the detectable shift in signal.

Embodiment 7: The nucleic acid sensor of embodiment 6, wherein the fluorophore and the quencher are on the same nucleic acid strand.

Embodiment 8: The nucleic acid sensor of embodiment 6, wherein the fluorophore and the quencher are on different nucleic acid strands.

Embodiment 9: The sensor of any one of embodiments 1-8, wherein each of the four separate nucleic acid strands is from about 30 to about 100 nucleotides.

Embodiment 10: The sensor of any one of embodiments 1-9, wherein the toehold domain is 13-19 nucleotides in length.

Embodiment 11: A composition comprising the nucleic acid sensor of any one of embodiments 1-10.

Embodiment 12: The composition of embodiment 11, wherein the composition further comprises at least one target nucleic acid, wherein the at least one target nucleic acid is complementary to the toehold domain of at least one of the two loops.

Embodiment 13: A kit comprising the nucleic acid sensor of any one of embodiment 1-10.

Embodiment 14: A method of detecting a target nucleic acid using the sensor of any one of embodiments 1-10, the composition of embodiment 11 or 12, or the kit of embodiment 13.

Embodiment 15: The method of embodiment 14, wherein the target nucleic acid is an intracellular nucleic acid, and wherein the target nucleic acid is detected without cell lysis.

Embodiment 16: The method of embodiment 15, wherein during use the sensor adopts a transmembrane conformation in which one portion of the sensor is inserted across the cell membrane into an intracellular location and wherein a different portion of the sensor is exposed at an extracellular location.

Embodiment 17: The method of embodiment 16, wherein a portion of the sensor containing the toehold domain complementary to the target nucleic acid is inserted across the cell membrane into the intracellular location and wherein a portion of the sensor containing the indicator is exposed at the extracellular location.

Embodiment 18: The method of embodiment 16, wherein a portion of the sensor containing the toehold domain complementary to the target nucleic acid and a portion of the sensor containing the indicator is inserted across the cell membrane into the intracellular location, and wherein a portion of the sensor containing the separate loop not containing the toehold domain is exposed at the extracellular location.

Embodiment 19: A sensor that adopts a transmembrane conformation, in which one portion of the sensor is on the luminal side of a lipid bilayer (membrane), and one portion is exposed on the extra-luminal side of the lipid bilayer.

Embodiment 20: A sensor that adopts a transmembrane conformation when introduced into the luminal or into the extra-luminal liquid media.

Embodiment 21: A sensor that resides in a transmembrane conformation for a period of time long enough to bind to a nucleic acid target.

Embodiment 22: A sensor that adopts an outward rectifying orientation, or an inward rectifying orientation, in which the sensor may or may-not contain design elements that perturbs the relative distribution of orientation which may include the addition of additional NA or other molecular motifs that favor, or dis-favor insertion across a lipid membrane.

Embodiment 23: A sensor in which when modified with DNA or RNA or other nucleic acid (NA) elements, including functional NA motifs designed to operate in isolation or in conjunction with other elements of the sensor, including binding of nucleic acid or other molecular targets, or small molecules, including fluorophores, quenchers will insert these elements across a lipid membrane.

Embodiment 24: A lipid membrane, from any one of embodiments 14, 15, 16, 17 and 18 may entail a synthetic lipid particle, including a vesicle, a biologically derived lipid membrane including an exosome, or an ex vivo cellular or tissue sample, a biological material including cell culture, or an in vivo organism

Embodiment 25: An assay including the “halo assay” in which fluorescence imaging is used to measure, characterize and optimize sensor insertion, target binding and signal amplification efficiencies by measuring a fluorescently tagged target molecule.

Embodiment 26: An assay including the “live-cell stability assay” in which fluorescence imaging is used to measure, characterize and optimize insertion efficiency, stability to biological factors, including endo-and exonucleases and orientation of a sensor by measuring the differential localization of fluorescent probes located on opposite ends of the sensor.

What is claimed is:

1. A nucleic acid sensor comprises:
(a) four separate nucleic acid strands,
wherein the four separate nucleic acid strands form a Holliday junction,
wherein portions of two of the four separate strands form two separate loops positioned at opposing ends of the sensor, and
wherein one of the two loops comprises a toehold domain complementary to a target nucleic acid:
(b) two of the four separate nucleic acid strands comprise a hydrophobic tag dividing the nucleic acid sensor into a shorter segment and a longer segment,
wherein the longer segment comprises the toehold domain: and
(c) one nucleic acid strand comprises an indicator that produces a detectable shift in signal upon binding of a target nucleic acid to the nucleic acid sensor.

2. The nucleic acid sensor of claim 1, wherein each of the four separate nucleic acid strands is from about 30 to about 100 nucleotides.

3. The nucleic acid sensor of claim 1, wherein the toehold domain is 13-19 nucleotides in length.

4. The nucleic acid sensor of claim 1, wherein the Holliday junction is a stacked Holliday junction wherein the two strands that form two separate loops intersect at the stacked Holiday junction and wherein the other two strands run parallel or antiparallel to each other.

5. The nucleic acid sensor of claim 1, wherein each hydrophobic tag is cholesterol.

6. The nucleic acid sensor of claim 1, wherein the indicator comprises a fluorophore.

7. The nucleic acid sensor of claim 6, wherein one nucleic acid strand comprises the fluorophore and one nucleic acid strand comprises a quencher, wherein the fluorophore and quencher are positioned such that a signal from the fluorophore is quenched in the absence of the target nucleic acid, and wherein binding of the target nucleic acid to the nucleic acid sensor results in strand displacement which releases the fluorophore from the quencher, thus producing the detectable shift in signal.

8. The nucleic acid sensor of claim 7, wherein the fluorophore and the quencher are on the same nucleic acid strand.

9. The nucleic acid sensor of claim 7, wherein the fluorophore and the quencher are on different nucleic acid strands.

10. The nucleic acid sensor of claim 1, wherein the target nucleic acid is an mRNA, miRNA, or IncRNA.

11. A nucleic acid sensor kit comprising four separate nucleic acid strands that are configured to form a Holliday junction,
wherein portions of a first nucleic acid strand and a second nucleic acid strand are configured to form two separate loops positioned at opposing ends of the sensor,
wherein each of a third nucleic acid strand and a fourth nucleic acid strand comprises a hydrophobic tag,
wherein one of the two loops comprises a toehold domain complementary to a target nucleic acid, and
wherein one of the third nucleic acid strand and the fourth nucleic acid strand comprises an indicator that is configured to produce a detectable shift in signal upon binding of a target nucleic acid to the nucleic acid sensor.

12. The nucleic acid sensor kit of claim 11 further comprising a neutralizing nucleic acid that is complementary to at least a portion of the target nucleic acid.

13. The nucleic acid sensor kit of claim 11, wherein the hydrophobic tag comprises cholesterol.

14. The nucleic acid sensor kit of claim 11, wherein each of the four separate nucleic acid strands is from about 30 to about 100 nucleotides.

15. A method of detecting a target nucleic acid enclosed within a vesicle or a cell comprising a lipid bilayer membrane, the method comprising:
incubating the vesicle or the cell with a nucleic acid sensor comprises:
(a) four separate nucleic acid strands,
wherein the four separate nucleic acid strands form a Holliday junction,
wherein portions of two of the four separate strands form two separate loops positioned at opposing ends of the sensor, and
wherein at least one of the two loops comprises a toehold domain complementary to a target nucleic acid:
(b) two of the four separate nucleic acid strands comprise a hydrophobic tag dividing the nucleic acid sensor into a shorter segment and a longer segment,
wherein the longer segment comprises the toehold domain: and
(c) one nucleic acid strand comprises an indicator that produces a detectable shift in signal upon binding of a target nucleic acid to the nucleic acid sensor: and
detecting the detectable shift in signal upon binding of the target nucleic acid to the longer segment of the nucleic acid sensor inside the vesicle or the cell.

16. The method of claim 15 further comprising incubating the vesicle or the cell with a neutralizing nucleic acid that is complementary to at least a portion of the target nucleic acid.

17. The method of claim 15, wherein the nucleic acid sensor adopts a transmembrane conformation, in which the longer portion of the sensor is on a luminal side of a lipid bilayer membrane, and a shorter portion is exposed on an extra-luminal side of the lipid bilayer membrane.

18. The method of claim 15, wherein the nucleic acid sensor adopts a transmembrane conformation, in which a shorter portion of the sensor is on a luminal side of a lipid bilayer membrane, and the longer portion is exposed on an extra-luminal side of the lipid bilayer membrane.

19. The method of claim 15, wherein the vesicle is an exosome.

20. The method of claim 15, wherein the vesicle is a cell-sized giant unilamellar vesicle.
